(19) United States
(12) Patent Application Publication
(10) Pub. No.: US 2026/0007821 A1
(43) Pub. Date: Jan. 8, 2026

(54) DRUG DELIVERY DEVICE AND SYSTEM

(71) Applicant: DAEGU GYEONGBUK INSTITUTE OF SCIENCE AND TECHNOLOGY, Daegu (KR)

(72) Inventors: Kyung In JANG, Daegu (KR); Hyeok Jun LEE, Daegu (KR)

(21) Appl. No.: 18/992,057

(22) PCT Filed: Sep. 2, 2022
(86) PCT No.: PCT/KR2022/013175

§ 371 Date: Jan. 7, 2025

(30) Foreign Application Priority Data
Jul. 26, 2022 (KR) 10-2022-0092700

(51) Int. Cl. A61M 5/145; A61M 5/168

(52) U.S. Cl. CPC A61M 5/145; A61M 5/16804; A61M 5/16877; A61M 2205/0216; A61M 2205/3334; A61M 2205/36

(57) ABSTRACT

One embodiment provides a drug delivery device comprising: a base part; a storage part in which drugs are stored inside; an expansion part which is disposed on the base part, has first cavities, and pressurizes the storage part; a heating part which is disposed between the base part and the expansion part and heats the first cavities; a flow path part which is disposed in the expansion part, has second cavities, and guides air inside the second cavities to the storage part; and a discharge part which discharges the drug to an injection target portion.

### TECHNICAL FIELD

The present invention relates to a device and system, more specifically to a drug delivery device and system.

### BACKGROUND ART

A drug delivery system is utilized in the fields of brain engineering and health care where drug treatment, behavior analysis, etc. are included. Conventional drug delivery systems are excellent in reliability, but pumps for injecting drugs are bulky in size and heavyweight, which makes it hard to be used in various environments in which miniaturization of a module is needed. In the case of conventional drug delivery systems that are miniaturized using flexible devices and thus utilized to study brain engineering, further, drugs are injected using thermally expanding polymers, so that a drug injection mechanism itself becomes disposable, thereby making it difficult to perform drug injection over a long period of time. Therefore, there is a definite need for development of a drug delivery system capable of being miniaturized, controlling a drug injection speed, and enabling drug injection over a long period of time.

### DISCLOSURE OF THE INVENTION

### Technical Problems

It is an object of the present invention to provide a drug delivery device and system that is capable of controlling a drug injection speed and a period of drug injection time, and if a drug is used up, being refilled with new drug.

The technical problems to be achieved through the present invention are not limited as mentioned above, and other technical problems not mentioned herein will be obviously understood by one of ordinary skill in the art through the following description.

### Technical Solutions

To achieve the above-mentioned objects, according to one aspect of the present invention, there is provided a drug delivery device including: a base part: a storage part for storing a drug therein; an expansion part disposed on top of the base part, having first cavities, and pressurizing the storage part thereagainst; a heating part disposed between the base part and the expansion part to heat the first cavities; a flow path part disposed on top of the expansion part, having second cavities, and guiding internal air of the second cavities to the storage part; and a discharge part for discharging the drug to an injection target.

According to the embodiment of the present invention, the heating part may include a heat generator and heat transferers, and the heat transferers may be arranged to face the first cavities in such a way as to heat the first cavities through the heat generated from the heat generator.

According to the embodiment of the present invention, the flow path part may be brought into close contact with the top of the expansion part to allow the second cavities to face the first cavities.

According to the embodiment of the present invention, the first cavities may be formed on the expansion part in such a way as to be arranged in series in a first direction, and the second cavities may be arranged in series to correspond to the first cavities in the first direction in such a way as to communicate with one another.

According to the embodiment of the present invention, one of the second cavities may have an inlet flow path for introducing air from the outside, and another second cavity may have an outlet flow path for guiding the air to the storage part.

According to the embodiment of the present invention, the expansion part may be made of a polydimethylsiloxane (PDMS) membrane.

To achieve the above-mentioned objects, according to another aspect of the present invention, there is provided a drug delivery system including: a drug delivery device for injecting a drug into a user's body; and a controller for controlling a drug injection speed of the drug delivery device, wherein the drug delivery device may include: a base part: a storage part for storing a drug therein; an expansion part disposed on top of the base part, having first cavities, and pressurizing the storage part thereagainst; a heating part disposed between the base part and the expansion part to heat the first cavities; a flow path part disposed on top of the expansion part, having second cavities, and guiding internal air of the second cavities to the storage part; and a discharge part for discharging the drug to an injection target.

According to the embodiment of the present invention, the heating part may include a heat generator and heat transferers, and the heat transferers may be arranged to face the first cavities in such a way as to heat the first cavities through the heat generated from the heat generator.

According to the embodiment of the present invention, the flow path part may be brought into close contact with the top of the expansion part to allow the second cavities to face the first cavities.

According to the embodiment of the present invention, the first cavities may be formed on the expansion part in such a way as to be arranged in series in a first direction, and the second cavities may be arranged in series to correspond to the first cavities in the first direction in such a way as to communicate with one another.

According to the embodiment of the present invention, one of the second cavities may have an inlet flow path for introducing air from the outside, and another second cavity may have an outlet flow path for guiding the air to the storage part.

According to the embodiment of the present invention, the expansion part is made of a PDMS membrane.

According to the embodiment of the present invention, the controller may allow the heating part to heat the first cavities so that the first cavities may expand to pressurize the second cavities.

According to the embodiment of the present invention, the controller may adjust a voltage intensity and a voltage application time to be applied to the heating part in such a way as to control an injection speed and time of the drug.

### Advantageous Effects of the Invention

According to the embodiments of the present invention, the drug delivery device and system can be miniaturized through the micro pump (that is, the expansion part) made of a flexible polymer. Further, the voltage intensity and voltage application time applied to the heating part can be adjusted to allow the expansion part to expand or be returned to its original shape, thereby controlling the drug injection speed and time, and if the stored drug is used up, refilling of the drug can be carried out to allow the drug delivery device to be reused, thereby enabling the injection of the drug over a long period of time.

The effectiveness of the invention is not limited as mentioned above, and it should be understood to those skilled in the art that the effectiveness of the invention may include another effectiveness as not mentioned above from the detailed description of the present invention.

### BRIEF DESCRIPTION OF DRAWINGS

FIG. 1 is a schematic view showing a drug delivery device and system according to an embodiment of the present invention.

FIG. 2 is an exploded perspective view showing the drug delivery device of FIG. 1.

FIG. 3 is an exploded view showing the drug delivery device at a different angle from an angle in FIG. 2.

FIG. 4 is a sectional view showing a state of the drug delivery device according to the embodiment of the present invention.

FIG. 5 is a sectional view showing another state of the drug delivery device according to the embodiment of the present invention.

FIG. 6 is a sectional view showing yet another state of the drug delivery device according to the embodiment of the present invention.

FIG. 7 is a sectional view showing still another state of the drug delivery device according to the embodiment of the present invention.

### BEST MODE FOR INVENTION

Hereinafter, the present invention will be described in detail below with reference to the accompanying drawings. However, the present invention may be implemented in different forms and should not be construed as limited to the embodiments set forth herein. In the drawings, reference numerals having no relation with the description will be omitted to allow the present invention to be clearly described, and the corresponding parts in the embodiments of the present invention are indicated by corresponding reference numerals.

When it is said that one element is described as being “connected” or “coupled” to the other element, one element may be directly connected or coupled to the other element, but it should be understood that another element may be present between the two elements. In contrast, when it is said that one element is described as being “directly connected” or “directly coupled” to the other element, it should be understood that another element is not present between the two elements.

Terms used in this application are used to only describe specific exemplary embodiments and are not intended to restrict the present invention. An expression referencing a singular value additionally refers to a corresponding expression of the plural number, unless explicitly limited otherwise by the context. In this application, terms, such as “comprise”, “include”, or ‘have”, are intended to designate those characteristics, numbers, steps, operations, elements, or parts which are described in the specification, or any combination of them that exist, and it should be understood that they do not preclude the possibility of the existence or possible addition of one or more additional characteristics, numbers, steps, operations, elements, or parts, or combinations thereof.

Now, an explanation of an embodiment of the present invention will be given in detail with reference to the attached drawings.

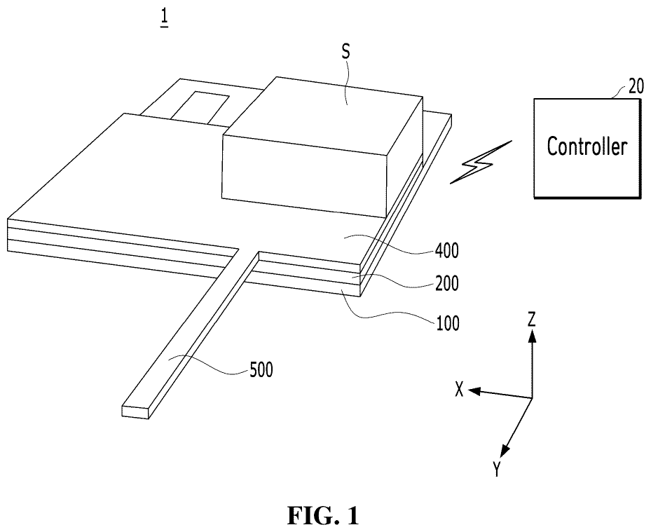

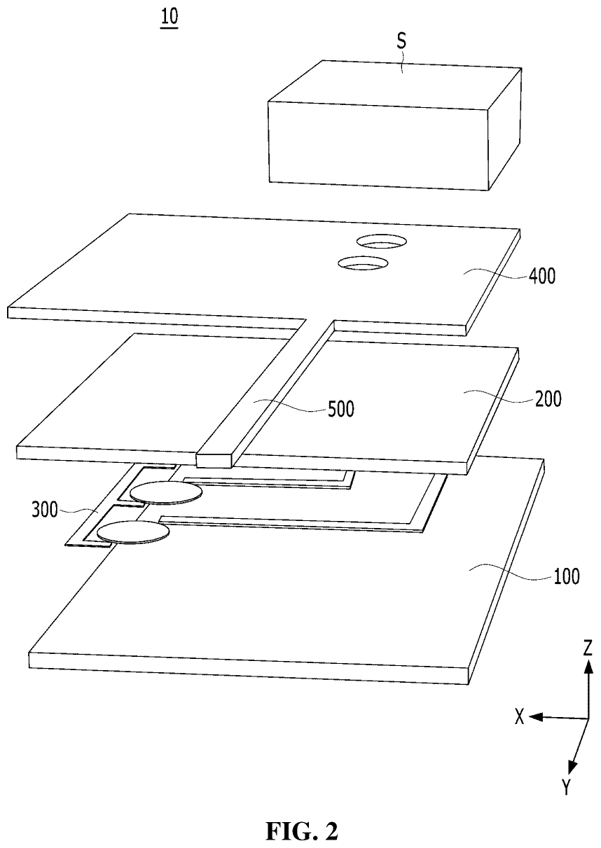

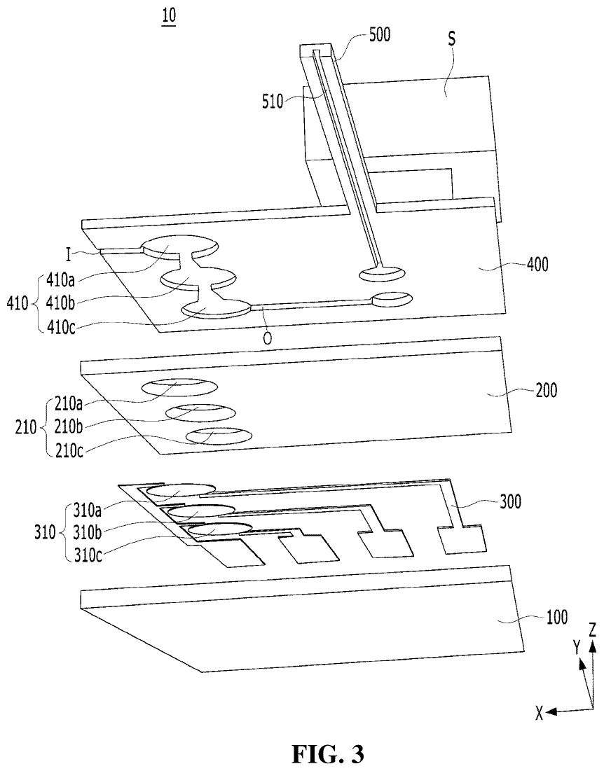

FIG. 1 is a schematic view showing a drug delivery device and system according to an embodiment of the present invention, and FIG. 2 is an exploded perspective view showing the drug delivery device of FIG. 1. FIG. 3 is an exploded view showing the drug delivery device at a different angle from an angle in FIG. 2.

Referring to FIGS. 1 to 3, a drug delivery system 1 is a system that is attached to a given area of a user (or patient)'s body to inject a drug into an injection target. In this case, the drug delivery system 1 includes a drug delivery device 10 and a controller 20.

The drug delivery device 10 is a device that injects the drug into the injection target through, for example, the pumping of a pump. In this case, the drug delivery device 10 includes a base part 100, an expansion part 200, a heating part 300, a flow path part 400, a discharge part 500, and a storage part S. In this case, the drug delivery device 10 is configured to allow the base part 100, the heating part 300, the expansion part 200, the flow path part 400, and the storage part S to be stacked on top of one another sequentially.

The base part 100 is a substrate on which the expansion part 200 and the heating part 300 are located. For example, the base part 100 may be a thin plate-shaped glass substrate, but the present invention may not be limited thereto.

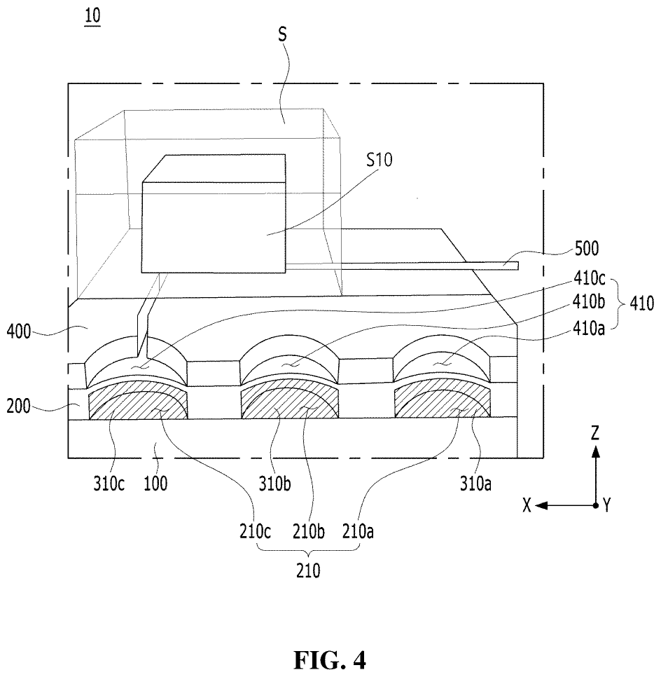

The storage part S stores the drug to be injected into the user. In detail, the storage part S has a storage space S10 in which the drug is received and stored (See FIGS. 4 to 7). The storage space S10 may be a housing located inside the storage part S. The storage space S10 communicates with the flow path part 400, which will be described in detail later.

If the drug stored in the storage space S10 is used up, the storage part S can be refilled with a new drug. According to an embodiment of the present invention, the storage space S10 has a refilling inlet (not shown) through which the drug is introduced therein from the outside. In this case, the refilling inlet is openable and closable. Further, if the drug stored in the storage space S10 is used up, the refilling inlet is open by the user or manager of the drug delivery device 10 to refill the storage space S10 with a new drug. If the refilling is completed, the refilling inlet is closed again to allow the drug to be injected into the user.

The expansion part 200 pressurizes the storage part S to allow the drug to be discharged from the storage space S10 if it is desired to inject the drug into the user. The expansion part 200 is made of an elastic material such as a polymer. For example, the expansion part 200 is made of a polydimethylsiloxane (PDMS) membrane.

The expansion part 200 is located on top of the base part 100. In detail, the expansion part 200 is stacked on a top surface of the base part 100. In this case, the top surface of the base part 100 represents one surface of the base part 100 that is located on an upper side with respect to a Z-axis in the drawings.

The expansion part 200 has first cavities 210. The first cavities 210 are concave grooves formed inwardly from one surface of the expansion part 200. In this case, one surface of the expansion part 200 is a surface (that is, the underside of the expansion part 200) facing the top surface of the base part 100 if the expansion part 200 is stacked on top of the base part 100. As a result, the open portions of the first cavities 210 are covered with the top surface of the base part 100.

One or more first cavities 210 are provided. As shown exemplarily in the drawings, three first cavities 210 are formed. For the brevity of the description, an embodiment in which the number of first cavities 210 is three will be explained below, but the present invention may not be limited thereto. The three first cavities 210 are designated dividedly as first cavities 210a, 210b, and 210c.

The first cavities 210 are formed on the expansion part 200 in such a way as to be arranged in series in a first direction. In this case, the first direction is a direction parallel to a longitudinal direction (to a direction of a Y-axis) of the expansion part 200. In detail, the first cavities 210a, 210b, and 210c are arranged in a line in the first direction (in the direction of the Y-axis).

The first cavities 210a, 210b, and 210c are spaced apart from one another at the same or similar intervals. In this case, the first cavities 210 are defined independently of one another. Accordingly, the first cavities 210a, 210b, and 210c do not communicate with one another.

The first cavities 210 have the same or similar shapes as or to one another. For example, the first cavities 210a, 210b, and 210c are cylindrical grooves having the same or similar sizes as or to one another.

The heating part 300 serves to heat at least a portion of the expansion part 200. In this case, the portion of the expansion part 200, which is heated by the heating part 300, may be the first cavities 210. According to an embodiment of the present invention, the heating part 300 generates heat through electric heating. In this case, the heating part 300 includes a heat generator (not shown) for generating the heat and heat transferers 310 for transferring the heat generated to the first cavities 210. In this case, the heating part 300 generates the heat through the power supplied from a power source part (not shown) located outside or inside the drug delivery device 10.

The heating part 300 is located between the base part 100 and the expansion part 200. In this case, the heat transferers 310 are arranged to face the first cavities 210. Accordingly, the heat transferers 310 are located between the first cavities 210 and the base part 100 covering the first cavities 210.

The heat transferers 310 have the same or similar shapes and sizes as or to the sections of the first cavities 210 that are viewed in a direction parallel to the expansion part 200 (e.g., in a direction parallel to an XY plane). As mentioned above, if the first cavities 210 are cylindrical grooves, the heat transferers 310 are circular plates having the same or similar diameters as or to the first cavities 210.

In this case, the expansion part 200 is brought into close contact with the base part 100. In this case, the underside of the expansion part 200 is brought into close contact with the top surface of the base part 100, while the heat transferers 310 are being received into the facing first cavities 210. Accordingly, the open portions of the first cavities 210 are covered with the base part 100 so that the first cavities 210 are sealed from the outside.

One or more heat transferers 310 are provided. As mentioned above, if the plurality of first cavities 210 are provided, the number of heat transferers 310 corresponds to the number of first cavities 210. In this case, the heat transferers 310 and the first cavities 210 are arranged face-to-face.

For example, if the expansion part 200 has the first cavities 210a, 210b, and 210c, the heating part 300 has a first heat transferer 310a, a second heat transferer 310b, and a third heat transferer 310c. In this case, the first heat transferer 310a, the second heat transferer 310b, and the third heat transferer 310c are located between the top surface of the base part 100 and the expansion part 200.

In this case, the first heat transferer 310a is arranged to face the first cavity 210a, and the second heat transferer 310b to face the first cavity 210b. Further, the third heat transferer 310c is arranged to face the first cavity 210c. With the heated generated from the heat generator, accordingly, the first heat transferer 310a heats the interior of the first cavity 210a, the second heat transferer 310b the interior of the first cavity 210b, and the third heat transferer 310c the interior of the first cavity 210c.

Like this, the heat transferers 310 corresponding to the first cavities 210 serve to heat at least some of the first cavities 210 at different times or at the same time, under the control of the controller 20 as will be discussed later.

The flow path part 400 serves to guide the air introduced from the outside to the storage part S. The flow path part 400 may be a microfluidic layer having micro flow paths along which air moves.

The flow path part 400 is stacked on a top surface of the expansion part 200. In this case, the top surface of the expansion part 200 is a surface facing the undersides of the first cavities 210. An underside surface of the flow path part 400 is brought into close contact with the top surface of the expansion part 200. In this case, the flow path part 400 includes an inlet flow path I, second cavities 410, and an outlet flow path O.

The second cavities 410 serve as paths that connect the inlet flow path I and the outlet flow path O to each other to guide the air introduced from the inlet flow path I to the outlet flow path O. The second cavities 410 are concave grooves formed inwardly from one surface of the flow path 400. In this case, one surface of the flow path part 400 is a surface (that is, the underside surface of the flow path part 400) facing the top surface of the expansion part 200 if the flow path part 400 is stacked on top of the expansion part 200. As a result, the open portions of the second cavities 410 are covered with the top surface of the expansion part 200.

One or more second cavities 410 are provided in such a way as to communicate with one another. As mentioned above, if the plurality of first cavities 210 are provided, the number of second cavities 410 corresponds to the number of first cavities 210. In this case, the second cavities 410 and the first cavities 210 are arranged face-to-face.

For example, if the expansion part 200 has the first cavities 210a, 210b, and 210c, the flow path part 400 has the second cavities 410a, 410b, and 410c. In this case, the second cavity 410a is arranged to face the first cavity 210a in an upward and downward direction (for example, in the direction of the Z-axis), and the second cavity 410b to face the first cavity 210b in the upward and downward direction (for example, in the direction of the Z-axis). Further, the second cavity 410c is arranged to face the first cavity 210c in the upward and downward direction (for example, in the direction of the Z-axis).

The plurality of second cavities 410 communicate with one another. The inlet flow path I is formed at one side of the second cavity 410a so that the second cavity 410a communicates with the outside, and the other side of the second cavity 410a communicates with one side of the first cavity 210b. The other side of the second cavity 410b communicates with one side of the second cavity 410c, and the outlet flow path O is formed at one side of the second cavity 410c so that the second cavity 410c communicates with the storage space S10. Accordingly, the air introduced through the inlet flow path I passes through the first cavities 210a to 210c and is then guided toward the storage space S10 through the outlet flow path O.

The discharge part 500 serves to discharge the drug stored in the storage part S to the injection target. The discharge part 500 protrudes outward from the storage part S. In this case, the discharge part 500 has a drug injection path 510 passing through an interior thereof in such a way as to extend to the storage part S. In this case, one end of the drug injection path 510 communicates with the storage space S10, and the other end is open to the outside of the discharge part 500.

According to an embodiment of the present invention, the discharge part 500 is located on the flow path part 400. In this case, the discharge part 500 protrudes outward from one side of the flow path part 400. In this case, one end of the drug injection path 510 is open to the outside of the discharge part 500. Further, the other end of the drug injection path 510 passes through the interior of the discharge part 500, extends to at least a portion of the flow path part 400, and is thus located on the underside of the storage space S10 in such a way as to communicate with the storage space S10.

The controller 20 controls a drug injection speed and time of the drug delivery device 10 into the injection target. In this case, the controller 20 represents a data processing device in hardware that has a physically structured circuit to implement functions expressed with codes or commands of a program. Examples of the data processing device in hardware include a microprocessor, a Central Processing Unit (CPU), a processor core, a multiprocessor, an Application Specific Integrated Circuit (ASIC), a Field-Programmable Gate Array (FPGA), etc., but the scope of the present invention may not be limited thereto.

According to an embodiment of the present invention, the controller 20 is disposed on a control device (not shown) provided separately from the drug injection device 10. In this case, the control device is a wireless device providing mobile communication, while ensuring portability and mobility, and includes all types of handheld wireless communication devices such as wireless handheld devices such as Personal Communication System (PCS), Global System for Mobile communications (GSM), Personal Digital Cellular (PDC), Personal Handyphone System (PHS), Personal Digital Assistant (PDA), International Mobile Telecommunication (IMT)-2000, Code Division Multiple Access (CDMA)-2000, W-Code Division Multiple Access (W-CDMA), Wireless Broadband Internet (WiBro) terminal, a smart phone, and the like and wearable devices such as a watch, a ring, a bracelet, an ankle bracelet, a necklace, glasses, a contact lens, a head-mounted device (HMD), and the like. In this case, the controller 20 generates a control signal under the control of the control device by the user and transmits the control signal to the drug delivery device 10 through wireless communication, thereby controlling the drug injection speed and time. However, the present invention may not be limited thereto, and otherwise, the controller 20 may be located on the drug delivery device 10. A method for controlling the drug delivery device 10 through the controller 20 will be described later.

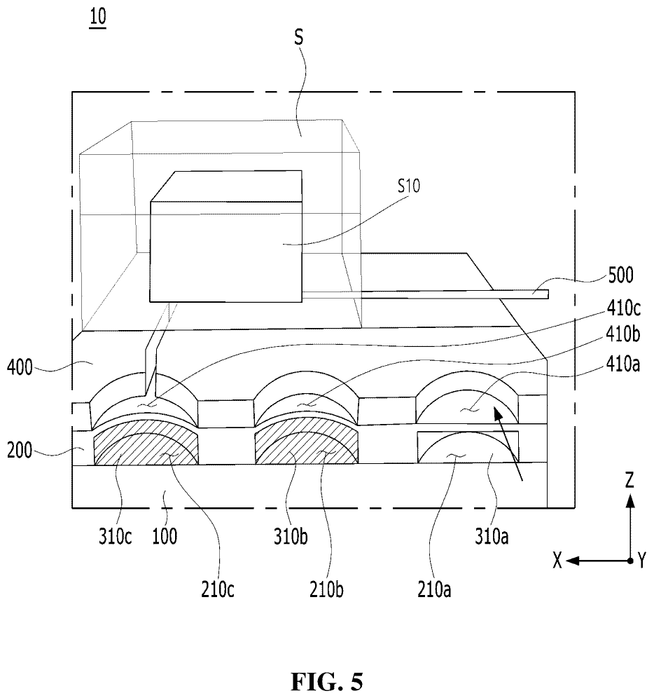

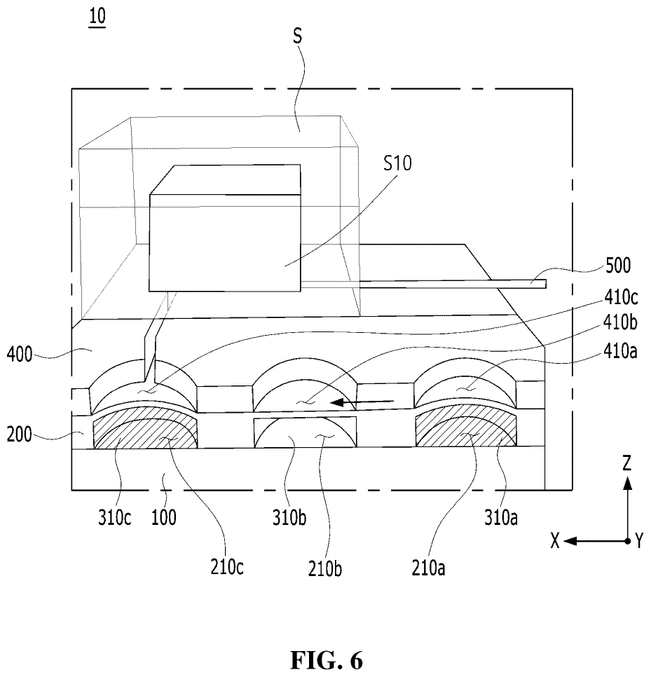

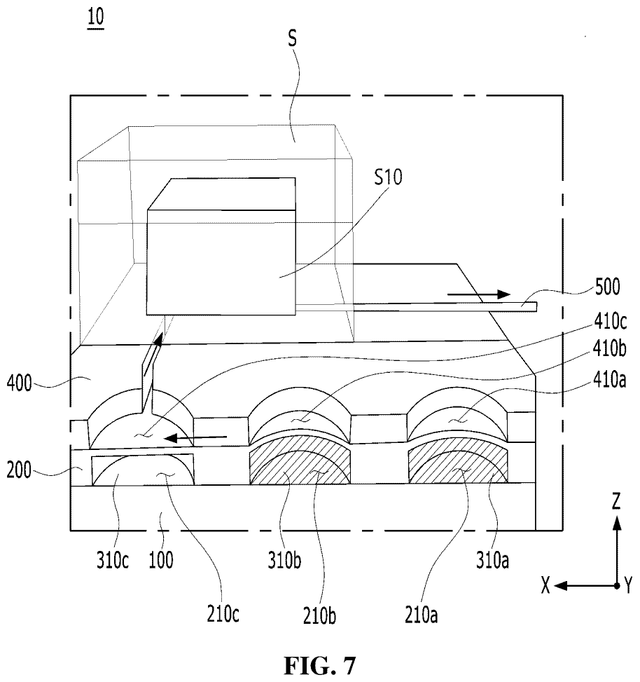

FIG. 4 is a sectional view showing a state of the drug delivery device according to the embodiment of the present invention. FIG. 5 is a sectional view showing another state of the drug delivery device according to the embodiment of the present invention. FIG. 6 is a sectional view showing yet another state of the drug delivery device according to the embodiment of the present invention. FIG. 7 is a sectional view showing still another state of the drug delivery device according to the embodiment of the present invention.

Referring to FIGS. 4 to 7, a method for injecting the drug into the user through the drug delivery device 10 and the drug delivery system 1 is carried out as follows. In this case, as mentioned above, the embodiment wherein three first cavities 210, three second cavities 410, and three heat transferers 310 are provided will be described, but the present invention may not be limited thereto.

First, as shown exemplarily in FIG. 4, the controller 20 allows the power source part to apply a voltage to the heating part 300. As a result, heat is generated from the heat generator and thus transferred to the heat transferers 310. The heat transferers 310 heat the facing first cavities 210.

In detail, the first heat transferer 310a heats the internal air of the first cavity 210a, and the second heat transferer 310b heats the internal air of the first cavity 210b. Further, the third heat transferer 310c heats the internal air of the first cavity 210c.

In this case, the internal air of the first cavities 210a, 210b, and 210c expands through the heating, so that internal pressures of the first cavities 210 being in a closed state increase. In this case, the internal pressures of the first cavities 210 are higher than internal pressures of the second cavities 410 not heated directly through the heating part 300, so that portions (which are close to the first cavities 210 and have relatively low thicknesses) of the expansion part 200 made of the elastic material deformedly expand upward (in the direction of the Z-axis). As a result, volumes of the first cavities 210a, 210b, and 210c become larger than volumes of the second cavities 410a, 410b, and 410c that face the first cavities 210a, 210b, and 210c. This causes a pressure to be generated in a direction from the first cavities 210 toward the second cavities 410, so that the second cavities 410 can be pressurized.

Next, as shown exemplarily in FIG. 5, the heat transfer to the first transferer 310a is stopped through the controller 20. As the heat transfer to the first transferer 310a is stopped, the first cavity 210a becomes cool so that the internal temperature of the first cavity 210a is lowered to allow the first cavity 210a to be returned to its original volume. As a result, the expansion part 200, which has expanded upward, is returned to its original shape to allow the pressure applied to the second cavity 410a to decrease. As the internal pressure decreases, a negative pressure in the second cavity 410a is generated. In this case, a difference between the internal and external pressures of the second cavity 410a is generated to allow external air to be introduced into the second cavity 410a through the inlet flow path I (See an arrow of FIG. 5).

Next, as shown exemplarily in FIG. 6, the heat transfer to the second heat transferer 310b is stopped to release a heating state through the controller 20. In this case, the heat transfer to the first heat transferer 310a restarts through the controller 20 to allow the interior of the first cavity 210a to be heated again. Further, the third heat transferer 310c keeps the heating state for the first cavity 210c.

In this case, as the internal temperature of the first cavity 210b becomes low, the first cavity 210b is returned to its original volume. Accordingly, the expansion part 200 close to the first cavity 210b is released from the expanding state thereof and thus returned to its original shape. As a result, the internal pressure of the second cavity 410b decreases to allow a negative pressure in the second cavity 410b to be generated. In this case, a pressure of the second cavity 410a increases through the re-heating for the first cavity 210a, so that the internal pressure of the second cavity 410a is higher than the internal pressure of the second cavity 410b. Accordingly, a difference between the internal pressures of the second cavities 410a and 410b is generated to allow the external air to move to the second cavity 410b (See an arrow of FIG. 6).

After that, as shown exemplarily in FIG. 7, the heat transfer to the third heat transferer 310c is stopped to release a heating state through the controller 20. In this case, the heat transfer to the second heat transferer 310b restarts through the controller 20 to allow the interior of the first cavity 210b to be heated again. Further, the first heat transferer 310a keeps the heating state for the first cavity 210a.

In this case, as the internal temperature of the first cavity 210c becomes low to allow the volume of the first cavity 210c to decrease, the expansion part 200 close to the first cavity 210c is released from the expanding state thereof. As a result, the internal pressure of the second cavity 410c decreases to allow a negative pressure in the second cavity 410c to be generated. In this case, a pressure of the second cavity 410b increases through the re-heating for the first cavity 210b, so that the internal pressure of the second cavity 410b is higher than the internal pressure of the second cavity 410c.

Accordingly, a difference between the internal pressures of the second cavities 410b and 410c is generated to allow the external air to pass through the second cavity 410b and the second cavity 410c and move toward the storage space S10. In this case, the air moving through the outlet flow path O pressurizes the storage space S10. As a result, the drug is discharged to the outside of the drug delivery device 10 through the discharge part 500 and thus injected into the user (See an arrow of FIG. 7).

The controller 20 controls the drug delivery device 10 to allow a drug injection cycle having the drug injection using the heating and cooling of the first cavities 210 to be performed at least one or more times by the drug delivery device 10, thereby injecting the drug into the user.

The controller 20 controls a drug injection speed and time through the adjustment of a voltage intensity and/or a period of voltage application time applied to the heating part 300.

According to an embodiment of the present invention, the controller 20 allows the voltage intensity or voltage application time applied to the heating part 300 to be differently controlled according to one time drug injection cycle (hereinafter, referred to as ‘first drug injection cycle’) and another time drug injection cycle (hereinafter, referred to as ‘second drug injection cycle’). For example, the controller 20 allows higher voltage in the second drug injection cycle than in the first drug injection cycle to be applied to the heating part 300. In this case, the expanding degrees of the first cavities 210 increase to allow the pressures applied to the second cavities 410 to increase. As a result, the flow rate of air passing through the second cavities 410a, 410b, and 410c and thus moving to the storage space S10 becomes fast to allow the injection speed of the drug discharged from the storage part S to increase. Contrarily, of course, it is possible that the controller 20 allows lower voltage in the second drug injection cycle than in the first drug injection cycle to be applied to the heating part 300.

For another example, the controller 20 allows the voltage in the second drug injection cycle to be applied to the heating part 300 for a longer period of time than that in the first drug injection cycle. In this case, the first cavities 210 in the second drug injection cycle have the same or similar expansion degrees as or to those in the first drug injection cycle, but they are kept in the expanding state for a longer period of time than those in the first drug injection cycle. As a result, pressures are applied to the second cavities 410 for the longer period of time, and accordingly, the flow of air passing through the second cavities 410a, 410b, and 410c and thus moving to the storage space S10 is kept longer. Contrarily, of course, it is possible that the controller 20 allows the voltage in the second drug injection cycle to be applied to the heating part 300 for a shorter period of time than that in the first drug injection cycle.

Further, the first drug injection cycle and the second drug injection cycle are performed continuously, but according to another embodiment of the present invention, after the first drug injection cycle is performed several times, the second first drug injection cycle is performed.

The present invention may not be limited to the above-mentioned embodiments, and the controller 20 adjusts the voltage intensity and/or voltage application time through various control methods to thus control the drug injection speed and time. In this case, the control of the drug delivery device 10 through the controller 20 is performed, for example, under the control of the control device by the user of the drug delivery device 10.

As described above, the drug delivery device 10 and the drug delivery system 1 according to the embodiments of the present invention can be miniaturized through the micro pump (that is, the expansion part 200) made of a flexible polymer. Further, the voltage intensity and voltage application time applied to the heating part 300 can be adjusted to allow the expansion part 200 to expand or be returned to its original shape, thereby controlling the drug injection speed and time, and if the stored drug is used up, refilling of the drug can be carried out to allow the drug delivery device 10 to be reused, thereby enabling the injection of the drug over a long period of time.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention. For example, the parts expressed in a singular form may be dispersedly provided, and in the same manner as above, the parts dispersed may be combined with each other.

It is therefore intended that the scope of the invention be limited not by this detailed description, but rather by the claims appended hereto, and it will be appreciated that the invention is intended to cover all modifications and equivalents within the scope of the following claims.

### INDUSTRIAL APPLICABILITY

The present invention is applicable in a drug delivery device and system in industrial fields.

1. A drug delivery device comprising:
a base part:
a storage part for storing a drug therein;
an expansion part disposed on top of the base part, having first cavities, and pressurizing the storage part thereagainst;
a heating part disposed between the base part and the expansion part to heat the first cavities;
a flow path part disposed on top of the expansion part, having second cavities, and guiding internal air of the second cavities to the storage part; and
a discharge part for discharging the drug to an injection target.

2. The drug delivery device according to claim 1, wherein the heating part comprises a heat generator and heat transferers, and the heat transferers are arranged to face the first cavities in such a way as to heat the first cavities through the heat generated from the heat generator.

3. The drug delivery device according to claim 2, wherein the flow path part is brought into close contact with the top of the expansion part to allow the second cavities to face the first cavities.

4. The drug delivery device according to claim 3, wherein the first cavities are formed on the expansion part in such a way as to be arranged in series in a first direction, and the second cavities are arranged in series to correspond to the first cavities in the first direction in such a way as to communicate with one another.

5. The drug delivery device according to claim 4, wherein one of the second cavities has an inlet flow path for introducing air from the outside, and another second cavity has an outlet flow path for guiding the air to the storage part.

6. The drug delivery device according to claim 1, wherein the expansion part is made of a polydimethylsiloxane (PDMS) membrane.

7. A drug delivery system comprising:
a drug delivery device for injecting a drug into a user's body; and
a controller for controlling a drug injection speed of the drug delivery device,
wherein the drug delivery device comprises:
a base part:
a storage part for storing a drug therein;
an expansion part disposed on top of the base part, having first cavities, and pressurizing the storage part thereagainst;
a heating part disposed between the base part and the expansion part to heat the first cavities;
a flow path part disposed on top of the expansion part, having second cavities, and guiding internal air of the second cavities to the storage part; and
a discharge part for discharging the drug to an injection target.

8. The drug delivery system according to claim 7, wherein the heating part comprises a heat generator and heat transferers, and the heat transferers are arranged to face the first cavities in such a way as to heat the first cavities through the heat generated from the heat generator.

9. The drug delivery system according to claim 8, wherein the flow path part is brought into close contact with the top of the expansion part to allow the second cavities to face the first cavities.

10. The drug delivery system according to claim 9, wherein the first cavities are formed on the expansion part in such a way as to be arranged in series in a first direction, and the second cavities are arranged in series to correspond to the first cavities in the first direction in such a way as to communicate with one another.

11. The drug delivery system according to claim 10, wherein one of the second cavities has an inlet flow path for introducing air from the outside, and another second cavity has an outlet flow path for guiding the air to the storage part.

12. The drug delivery system according to claim 7, wherein the expansion part is made of a PDMS membrane.

13. The drug delivery system according to claim 9, wherein the controller allows the heating part to heat the first cavities so that the first cavities expand to pressurize the second cavities.

14. The drug delivery system according to claim 10, wherein the controller adjusts a voltage intensity and a voltage application time to be applied to the heating part in such a way as to control an injection speed and time of the drug.
